(19) United States
(12) Patent Application Publication
(10) Pub. No.: US 2024/0194301 A1
(43) Pub. Date: Jun. 13, 2024

(54) SYSTEMS AND METHODS TO EVALUATE INACTIVATION KINETICS OF A BIOLOGICAL INDICATOR

(71) Applicant: AMGEN INC., Thousand Oaks, CA (US)

(72) Inventors: Nicholas DeSantis, Thousand Oaks, CA (US); Shaun Devitt, King of Prussia, PA (US)

(21) Appl. No.: 18/286,242

(22) PCT Filed: May 18, 2022
(86) PCT No.: PCT/US22/29796

§ 371 Date: Oct. 10, 2023

Related U.S. Application Data
(60) Provisional application No. 63/189,934, filed on May 18, 2021.

(51) Int. Cl. G16C 20/10; C12Q 1/22

(52) U.S. Cl. CPC G16C 20/10; C12Q 1/22

(57) ABSTRACT

Systems and methods are provided for evaluate inactivation kinetics of a biological indicator using a resistometer that is intended for a gaseous sterilization process. The gaseous sterilization process may include multiple sterilant gas injection phases. Systems and methods are provided for evaluating correlation of D values, obtained by both enumeration and fraction negative methods for determining viable spore counts, and to determine whether there are any serious deviations from a linear survivor curve that is based on the enumeration and fraction negative methods.

### CROSS-REFERENCE TO RELATED APPLICATION

Priority is claimed to U.S. Provisional Application No. 63/189,934, filed May 18, 2021, the entire contents of which are hereby incorporated by reference herein.

### FIELD OF DISCLOSURE

The present disclosure relates to evaluation of inactivation kinetics of a biological indicator using a resistometer that is configured to implement a gaseous sterilization process that includes multiple sterilant gas injection phases.

### BACKGROUND

Biological indicators (BIs) are devices containing viable microorganisms with typical total population on a magnitude of greater than or equal to 10⁶ spores that, for example, may be intended to provide defined resistance to a specified sterilization process. The devices may be used to, for example, demonstrate that a given sterilization process can achieve a desired sterility assurance level for a given product.

To evaluate the resistance of biological indicators, the biological indicators may be, for example, placed in a resistometer chamber, and subject to incremental exposure time conditions for a gaseous sterilant. Distinct groups of biological indicators are exposed independently to a sequence of increasing exposure times and evaluated to determine the response of surviving population versus the exposure time. For shorter exposure times, individual biological indicators contain hundreds or thousands of surviving microorganisms, and the survival count may be determined through the standard direct enumeration method. For longer exposure times, individual biological indicators may contain a small number of or even no surviving microorganisms, and so the survival count may be determined through growth/no growth BI sterility testing followed by a calculation of most probable number (MPN) of survivors from the fraction negative results.

Per ISO 14937, an overkill approach for gaseous sterilization processing validation requires the use of biological indicators with a microorganism population of 10⁶ spores or higher that are demonstrably more resistant than the bioburden of the product. In this approach, BIs are often placed in a most difficult to sterilize location within a device, and are subject to a gaseous sterilization cycle exhibiting half the sterilant gas exposure time conditions as compared to the commercial sterilization cycle. Under these “half cycle” conditions, a goal may be to inactivate the biological indicator, and thereby achieving a 6-spore log reduction for an associated biological indicator organism. Once a 6-spore log reduction is achieved, a resulting inactivation profile of the biological indicator organism may be extrapolated, and demonstrate that in a “full cycle” with double the exposure conditions, a 12-spore log reduction may be achieved for the biological indicator organism.

Known extrapolation approaches may be best suited for gaseous sterilizing processes that exhibit linear inactivation kinetics for the biological indicators (e.g., ethylene oxide sterilization processes, steam sterilization processes, etc.), as that linearity may support the justification for extrapolation to 12 log reduction for the “full cycle” process by simply doubling the exposure conditions of the “half cycle”. Using enumeration and/or fraction negative MPN methods to determine viable spore counts remaining on each biological indicator test sample, an inactivation profile of the biological indicator for the specific sterilization method can be assessed and established. For many traditional gaseous sterilization processes (e.g., ethylene oxide, moist heat, etc.) inactivation kinetics of an associated biological indicator may be readily shown to be linear using, for example, methods as prescribed in ISO 11138-1.

Surface sterilization modalities often utilize multiple sets of gas injection/dwell phases, or “gas charges,” instead of a single gas charge per a commercial or production cycle. Therefore, traditional approaches for measuring resistance of the biological indicator using resistometer cycles with incremental exposure dwell times and all other parameters constant (and therefore only utilizing a single gas injection/dwell event per run) may not be the most appropriate method for use to extrapolate from “half” cycle to “full” cycle conditions for sterilization processes that employ multiple gas charges per production sterilization “full cycle” run. Currently no comprehensive approach exists for utilizing resistometer cycles that incorporate multiple sterilant gas charges during processing to establish resistance characteristics and demonstrate continued linearity and maintenance of D Value through multiple/additional gas charges for biological indicators in a gaseous sterilization process.

Apparatuses, systems, and methods are needed to evaluate inactivation kinetics of biological indicators that are subjected to gaseous sterilizing processes which exhibit non-linear inactivation kinetics for the biological indicators. Apparatuses, systems, and methods are needed to evaluate inactivation kinetics of a biological indicator using a resistometer that is configured to implement a gaseous sterilization process. Apparatuses, systems, and methods are also needed to evaluate correlation of D values.

### SUMMARY

A computer-implemented method may evaluate inactivation kinetics of a biological indicator population, under sterilization processing conditions of increasing sterilant exposure, using data obtained from a resistometer that is configured to implement a gaseous sterilization process. The method may include a first study arm in which there is processing, using the resistometer or other chamber (i.e., developmental), of biological indicator test groups under cycle parameters providing for at least one first sterilant gas injection phase. The method may also include calculating D Value using an enumeration method for determining viable spore counts for processed BIs at incremental exposure conditions under processing conditions of at least one sterilant gas injection phase. The method may also include using processed BI sterility test results and fraction negative method for determining viable spore counts under processing conditions of at least one first sterilant gas injection phase. The method may further include a second or additional study arm(s) in which there is processing, using the resistometer or other chamber (i.e., developmental), of biological indicator test groups under cycle parameters providing for at least two sterilant gas injection phases and at least one additional sterilant injection phase compared to the first set of exposure conditions as described above. The method may also include using enumeration and/or BI sterility test results and fraction negative method for determining viable spore counts for BIs processed under cycle parameters providing for at least two sterilant gas injection phases and at least one additional sterilant injection phase compared to the first study arm processing conditions as described above. The method may also include establishing an inactivation profile of the biological indicator based on the combined viable spore counts determined in multiple study arms as described above.

In another embodiment, a computer-implemented method may calculate a D value from viable spore count data obtained from each study arm described above. The method may include determining D values from viable spore counts determined from the enumeration method. The method may also include establishing a survivor curve and calculating D values via viable spore counts obtained through fraction negative method or obtained from enumeration. This method may also include evaluating linearity of the survivor curve determined using viable spore count data obtained either from fraction negative or enumeration methods for each study arm described above. The method may further include establishing a combined survivor curve using the survival curves determined from multiple study arms as described above. The method may yet further include determining biological indicator D value, and evaluating linearity of the combined survivor curve as described above.

In a further embodiment, a computer-readable medium may store computer-readable instructions that, when executed by a processor, may cause the processor to construct a survivor curve and/or calculate D Values and/or construct survival curves and/or evaluate inactivation kinetics and/or assess the linearity of a survivor curve of a biological indicator processed under incremental faseous sterilization processing conditions using viable spores count data obtained from resistometer studies.

In a further embodiment, a computer-readable medium may store computer-readable instructions that, when executed by a processor, may cause the processor to construct a survivor curve(s) and/or calculate D Values and/or evaluate inactivation kinetics and/or assess the linearity of a survivor curve of a biological indicator processed under incremental gaseous sterilization processing conditions using a viable spores count data obtained from resistometer studies utilizing one or additional sterilant gas injection phase(s) from one or multiple study arms collectively as described above.

In another embodiment, a computer-readable medium may store computer-readable instructions that, when executed by a processor, may cause the processor to construct a survivor curve(s) and/or calculate D Values and/or evaluate inactivation kinetics and/or assess the linearity of a survivor curve of a biological indicator processed under incremental gaseous sterilization processing conditions using viable spores count data obtained from resistometer studies utilizing one or additional sterilant gas injection phase(s) from subsequent study arms as described above.

In a further embodiment, a system may evaluate correlation of D values derived from multiple survival curves constructed from multiple study arms as described above.

In another embodiment, a system may evaluate linearity of a survival curve constructed from multiple survival curves from multiple study arms as described above, and demonstrate continued linearity and maintenance of D Value for the BI through multiple/additional gas charges..

A computer-implemented method may evaluate inactivation kinetics of a biological indicator using data obtained from results of studies executed in a resistometer that is configured to implement a gaseous sterilization process. The computer-implemented method may be within the software of a computer housed within or that communicates with a resistometer computer system (i.e., a remote computing device). The method may include receiving a first set of biological indicator data from studies using biological indicators processed in the resistometer with sterilization exposures consisting of at least one first sterilant gas injection phase. The first set of biological indicator data may be representative of viable spore counts of the biological indicator. The method may also include determining enumeration-D values data based on the first biological indicator data obtained via using an enumeration method for determining viable spore counts. The method may further include receiving a second set of biological indicator data from studies using virological indicators process in the resistometer with sterilization exposure consisting of at least a second sterilant gas injection phase. The second biological indicator data may be representative of viable spore counts of the biological indicator. The method may yet further include determining D-Value and/or performing survivor curve analysis based on the second set of biological indicator data, obtained via the fraction negative method to determine viable spores counts. The method may also include establishing an inactivation profile of the biological indicator based on the enumeration-D values data and the fraction-negative-D values data obtained from the first and second sets of biological indicator data.

In another embodiment, a computer-implemented method may evaluate correlation of D values. The method may include determining D values using viable spore counts determined via an enumeration method, and determining an enumeration-linear survivor curve based on the enumeration-D values. The method may also include determining D values using viable spore counts determined via a fraction negative method, and determining a fraction-negative-linear survivor curve based on the fraction-negative-D values. The method may further include establishing a combined linear biological indicator survivor curve based on the enumeration-linear survivor curve and the fraction-negative-linear survivor curve. The method may yet further include determining biological indicator D value deviations from the combined linear survivor curve.

In a further embodiment, a computer-readable medium may store computer-readable instructions that, when executed by a processor, may cause the processor to evaluate inactivation kinetics of a biological indicator using data from studies that utilized a resistometer configured to implement a gaseous sterilization process. The computer-readable medium may include a first set of biological indicator data receiving module that, when executed by a processor, may cause the processor to receive a first set of biological indicator data obtained from test samples processed under at least one first sterilant gas injection phase. The first set of biological indicator data may be representative of viable spore counts of the biological indicator. The computer-readable medium may also include an enumeration-D values data generation module that, when executed by a processor, may cause the processor to generate D values from enumberation viable spores counts data based on the first set of biological indicator data. The computer-readable medium may further include a second set of biological indicator data receiving module that, when executed by a processor, may cause the processor to receive a second set of biological indicator data obtained from test samples processed under at least one additional sterilant gas injection phase. The second set of biological indicator data may be representative of viable spore counts of the biological indicator. The computer-readable medium may yet further include a fraction-negative-D values data generation module that, when executed by a processor, may cause the processor to generate D values from fraction negative viable spores counts data based on the second set of biological indicator data. The computer-readable medium may also include an inactivation profile data generation module that, when executed by a processor, may cause the processor to establish an inactivation profile of the biological indicator based on the enumeration-D values data and the fraction-negative-D values data.

In yet a further embodiment, a computer-readable medium may store computer-readable instructions that, when executed by a processor, may cause the processor to evaluate correlation of D values with the D Values generated from different sets of data. The computer-readable medium may include an enumeration-D values data determination module that, when executed by a processor, may cause the processor to determine enumeration-D values data via viable spores counts determined from an enumeration method. The computer-readable medium may also include an enumeration-linear survivor curve determination module that, when executed by a processor, may cause the processor to determine an enumeration-linear survivor curve based on enumeration viable spores counts data. The computer-readable medium may further include a fraction-negative-D values determination module that, when executed by a processor, may cause the processor to determine D values based on viable spores counts data determined via a fraction negative method. The computer-readable medium may yet further include a fraction-negative-linear survivor curve determination module that, when executed by a processor, may cause the processor to determine a fraction-negative-linear survivor curve based on the viable spores counts data determined from a fraction negative method. The computer-readable medium may also include a linear biological indicator survivor curve establishment module that, when executed by a processor, may cause the processor to establish a linear biological indicator survivor curve based on viable spores counts data determined via enumeration and viable spores counts data determined via a fraction-negative method. The computer-readable medium may further include an inactive profile deviation determination module that, when executed by a processor, may cause the processor to determine biological indicator inactive profile deviations from the linear survivor curve.

In another embodiment, a system may evaluate inactivation kinetics of a biological indicator using a resistometer configured to implement a gaseous sterilization process. The system may include a time clock, a resistometer chamber pressure sensor, a resistometer chamber temperature sensor, a resistometer chamber relative humidity sensors, and at least one resistometer chamber sterilant concentration level sensor. The system may include a first set of biological indicator data receiving module stored on a memory that, when executed by a processor, may cause the processor to receive a first set of biological indicator data during at least one first sterilant gas injection phase. The first set of biological indicator data may be representative of viable spore counts of the biological indicator. The system may also include a D values data generation module stored on a memory that, when executed by a processor, may cause the processor to generate enumeration-D values data, implementing an enumeration method for determining viable spore counts, based on the first set of biological indicator data. The system may further include a second set of biological indicator data receiving module stored on a memory that, when executed by a processor, may cause the processor to receive a second set of biological indicator data during at least one second sterilant gas injection phase. The second set of biological indicator data may be representative of viable spore counts of the biological indicator. The system may yet further include a fraction-negative-D values data generation module stored on a memory that, when executed by a processor, may cause the processor to generate fraction-negative-D values, implementing an fraction negative method for determining viable spore counts, based on the second biological indicator data. The system may also include an inactivation profile data generation module stored on a memory that, when executed by a processor, may cause the processor to establish an inactivation profile of the biological indicator based on the enumeration-D values and the fraction-negative-D values.

In a further embodiment, a system may evaluate correlation of D values. The system may include at least one resistometer chamber sterilant injection mechanism and at least one resistometer chamber sterilant concentration level sensor. The system may include an enumeration-D values determination module stored on a memory that, when executed by a processor, may cause the processor to determine enumeration-D values via an enumeration method for determining viable spore counts. The system may also include an enumeration-linear survivor curve determination module stored on a memory that, when executed by a processor, may cause the processor to determine an enumeration-linear survivor curve based on the enumeration-D values. The system may further include a fraction-negative-D values determination module stored on a memory that, when executed by a processor, may cause the processor to determine fraction-negative-D values via a fraction negative method for determining viable spore counts. The system may yet further include a fraction-negative-linear survivor curve determination module stored on a memory that, when executed by a processor, may cause the processor to determine a fraction-negative-linear survivor curve based on the fraction-negative-D values. The system may also include a linear biological indicator survivor curve establishment module stored on a memory that, when executed by a processor, may cause the processor to establish a linear biological indicator survivor curve based on the enumeration-linear survivor curve and the fraction-negative-linear survivor curve. The system may further include an enumeration-D values determination module stored on a memory that, when executed by a processor, may cause the processor to determine biological indicator D value deviations from the linear survivor curve.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicated of the presence or absence of particular elements in any of the exemplary embodiments, except as may be explicitly delineated in the corresponding written description. Also, none of the drawings are necessarily to scale.

FIG. 1A depicts an example system to evaluate inactivation kinetics of a biological indicator using a resistometer that is configured to implement a gaseous sterilization process that includes multiple sterilant gas injection phases;

FIG. 1B depicts a high-level block diagram of a system to evaluate inactivation kinetics of a biological indicator;

FIGS. 1C-1G depict high-level block diagrams of various example apparatuses and methods for use within a system to evaluate inactivation kinetics of a biological indicator;

FIG. 2 depicts a graph of an inactivation profile for a biological indicator using viable spore counts data obtained from, for example, a direct enumeration method and, for example, a fraction-negative method from a resistometer study that utilized processing conditions of a single sterilant gas injection phase; and

FIGS. 3A and 3B depict graphs of an inactivation profile for a biological indicator using combined viable spore counts data obtained from, for example, the direct enumeration method and a fraction-negative method from a resistometer study that utilized processing condition of multiple sterilant gas injection phases;

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercial feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Apparatuses, systems, and methods are provided to evaluate inactivation kinetics of biological indicators (BIs) that may be subjected to gaseous sterilizing processes which offer challenges when evaluating inactivation kinetics for the biological indicators. Apparatuses, systems, and methods are provided to evaluate inactivation kinetics of a biological indicator using a resistometer that is configured to implement a gaseous sterilization process. Apparatuses, systems, and methods are also provided to evaluate correlation of D values. A “D value” is generally understood to be a time to reduce a BI population by one log under certain sterilization processing conditions.

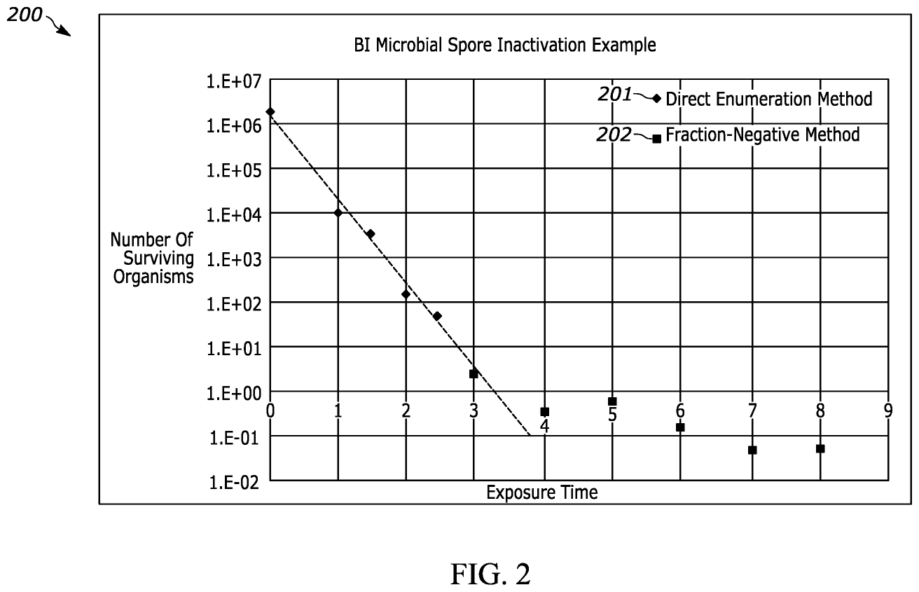

For some gaseous sterilization modalities (e.g., a nitrogen dioxide modality, a hydrogen peroxide modality, a “surface” sterilization modality, etc.), challenges may exist in defining areas of an associated biological indicator (BI) survival curve (e.g., survival curve 200 of FIG. 2, etc.) using common methods (i.e., specifically for the areas as outlined in regions 2 and 3 per ISO 11138-7:2019, FIG. A. 4 of ISO 11138-7:2019, regions 201 and 202 of FIG. 2).

As described in detail herein, biological indicator (BI) survival curves may be generated for gaseous sterilization modalities, there may be common inherent differences in the sterilization process (e.g., generating a biological indicator (BI) survival curve using a sterilization process in which a sterilant gas is introduced into a resistometer chamber to maintain sterilant concentration levels during an associated production sterilization cycle, etc.).

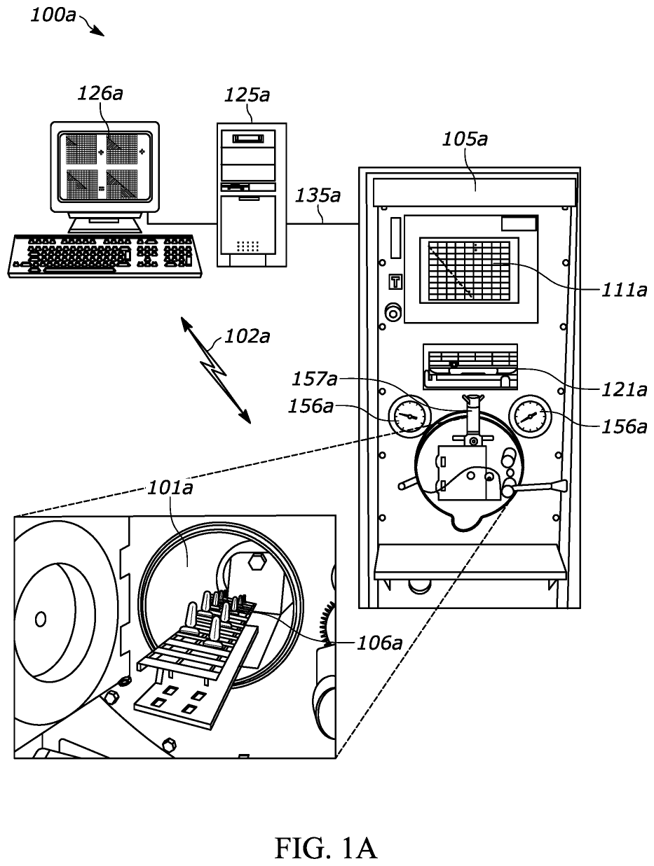

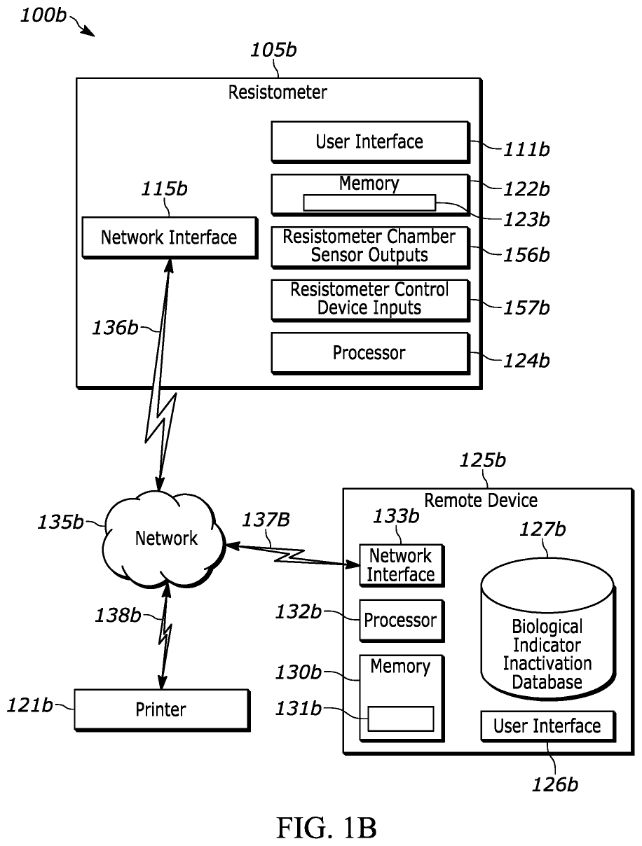

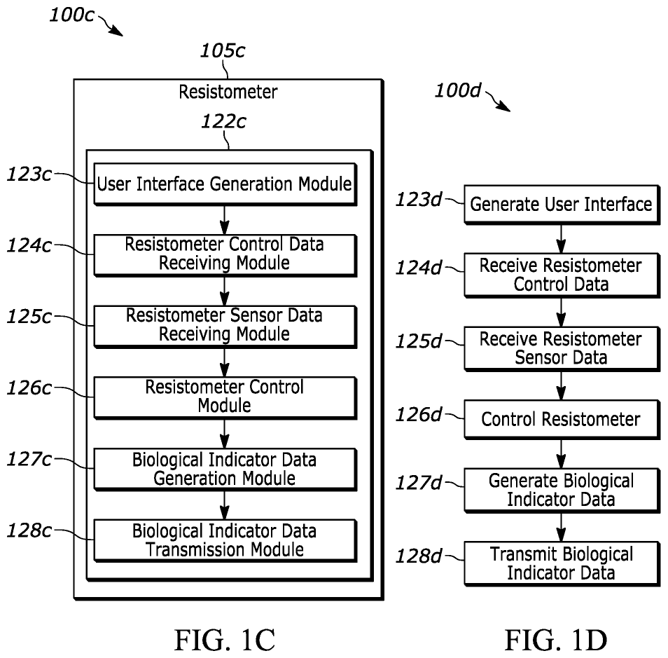

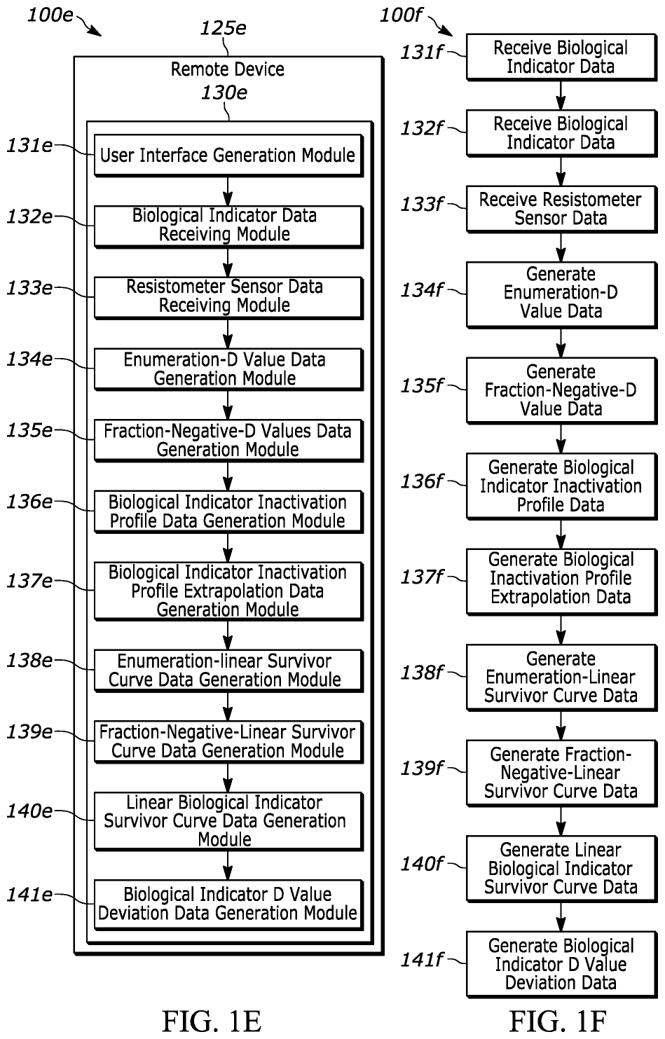

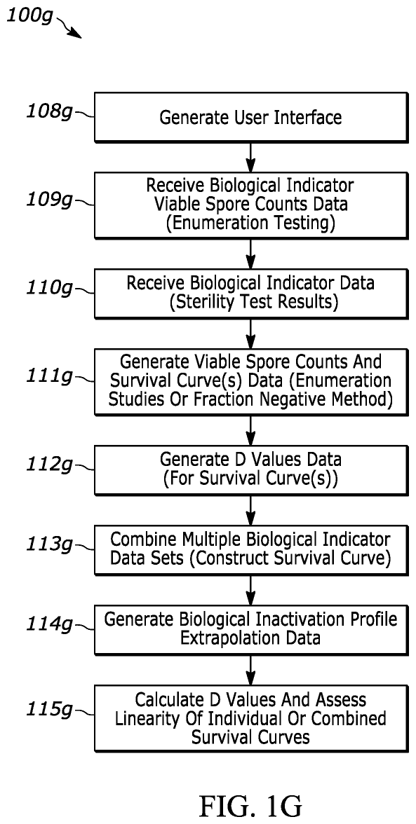

Turning to FIG. 1A, a system to evaluate inactivation kinetics of a biological indicator 100a may include a resistometer 105a that may be, for example, configured to implement a gaseous sterilization process that includes multiple sterilant gas injection phases. Details of a specific implementation of a system to evaluate inactivation kinetics of a biological indicator 100a are included herein with reference to sterilization process including a nitrogen dioxide (NO₂) modality. Other implementations of the system 100a may be directed to, for example, other sterilization processes associated challenges in characterizing survival curves generated from resistometer studies.

BIs may be removed from the resistometer chamber and then sent to a micro lab for sterility testing. The results of the micro lab sterility tests may be manually entered in a manual calculation method or software calculation method to complete the enumeration/fraction positive assessments, and to calculate D Values/linearity/inactivation profile. Suggest we meet to discuss how this impacts these sections and overall application/strategy. Alternatively, or additionally, viable spore counts data may be obtained from enumeration and Fraction negative tests, and from that data it provides individual or combined survival curve, D Value, linearity information for the BI.

The system 100a may also include a remote device 125a (e.g., a remote computing device, etc.) communicatively connected to the resistometer 105a via, for example, a network 135a. As described in detail herein, a user interface displayed of, for example, a display (e.g., display 111a and/or 126a of FIG. 1A, etc.) of a resistometer 105a and/or a remote device 125a, respectively, may be configured to enable a user to evaluate inactivation kinetics of a biological indicator.

The resistometer 105a may include at least one resistometer chamber 101a configured to, for example, receive a plurality of biological indicators (BIs) 106a. The resistometer 105a may include at least first and second resistometer chamber sensor outputs 156a (e.g., a resistometer chamber pressure, a resistometer chamber temperature, a resistometer chamber relative humidity sensor, a resistometer chamber sterilant concentration level sensor, a combination thereof, a sub-combination thereof, etc.), at least one resistometer control device output 157a (e.g., a sterilant gas injection control device output, a temperature control device output, a pressure control device output, etc.), a printer 121a and a system clock for both controlling programmed inputs and reporting outputs.

With reference to FIG. 1B, a system to evaluate inactivation kinetics of a biological indicator 100b may include a resistometer 105b, a printer 121b, and a remote device 125b communicatively interconnected via a network 135b. The system to evaluate inactivation kinetics of a biological indicator 100b may be similar to, for example, the system to evaluate inactivation kinetics of a biological indicator 100a of FIG. 1A. The resistometer 105b may be similar to, for example, the resistometer 105a of FIG. 1A. The remote device 125b may be similar to, for example, the remote device 125a of FIG. 1A. The printer 121b may be similar to, for example, the printer 121a of FIG. 1A. The network 135b may be similar to, or communicatively connected to, for example, the network 135a of FIG. 1A.

A resistometer 105b may include a memory 122b and a processor 121b for storing and executing, respectively, a module 123b. The module 123b, stored in the memory 122b as a set of computer-readable instructions, may be related to an application for implementing at least a portion of the system for evaluate inactivation kinetics of a biological indicator 100b. As described in detail herein, the processor 124b may execute the module 123b to, among other things, cause the processor 124b to receive, generate, and/or transmit data (e.g., biological indicator data, etc.) with the remote device 125b, and/or the printer 121b.

The resistometer 105b may also include a user interface 111b which may be any type of electronic display device, such as touch screen display, a liquid crystal display (LCD), a light emitting diode (LED) display, a plasma display, a cathode ray tube (CRT) display, or any other type of known or suitable electronic display along with a user input device. A user interface 111b may exhibit a user interface display (e.g., any user interface 111a, 126a of FIG. 1A, any of the graphs of FIGS. 2-5B, etc.) which may, for example, depict a user interface for implementation of at least a portion of the system for evaluate inactivation kinetics of a biological indicator 100b.

The resistometer 105b may also include at least one resistometer chamber sensor output 156b, at least one resistometer control device input 157b, and a network interface 115b. The network interface 115b may be configured to facilitate communications, for example, between the resistometer 105b and the network 135b via any wireless communication network 136b, including for example: a wireless LAN, MAN or WAN, WiFi, TLS v1.2 WiFi, the Internet, or any combination thereof. Moreover, a resistometer 105b may be communicatively connected to any other device via any suitable communication system, such as via any publicly available or privately owned communication network, including those that use wireless communication structures, such as wireless communication networks, including for example, wireless LANs and WANs, satellite and cellular telephone communication systems, etc.

The resistometer 105b may include a clock (e.g., a digital clock, etc.) capable of, for example, ‘Time Resolution’ of 00:00.01 and accuracy of + or −00:00:02. The resistometer 105b may include a resistometer chamber temperature sensor having a temperature resolution of, for example, 0.1 C and accuracy of 0.5 C. The resistometer 105b may include a resistometer chamber pressure sensor having a pressure accuracy of, for example, 0.5 psia. In the case of NO₂ sterilization, a far higher precision and accuracy for a resistometer chamber pressure sensor 105b may include precision on an order of 0.01 psia, and an accuracy on an order of 0.1 psia. The resistometer 105b may include a resistometer chamber temperature sensor having a temperature control of, for example, + or −0.5 C during exposure. The resistometer 105b may include a resistometer chamber vacuum level capable of, for example, 0.65 psia. The resistometer 105b may include a resistometer chamber steam charge time from, for example, 100° ° C. to set temperature within, for example, 10 seconds or less. The resistometer 105b may include a resistometer chamber relative humidity sensor having a relative humidity control, for example, +/−5% during exposure. The resistometer 105b may include a resistometer chamber sterilant concentration level sensor (e.g., a nitrogen dioxide (NO₂) sensor, a hydrogen peroxide (H₂O₂) sensor, an ethylene oxide (C₂H₄O) sensor, a moist heat sensor, etc.).

The resistometer 105b may include a “trap door” element of the resistometer chamber 101a. The resistometer chamber 101a may contain a small metal or plastic basket intended to hold BI test samples that could be lowered out of the main resistometer chamber 101a through a chamber wall door and into a separate air washing lower chamber, with the door closing and sealing the main chamber from the lower chamber, in which the BI test samples may be lowered after, for example, a sterilant gas evacuation phase and, if desired, after each gas charge. The basket may be reintroduced into the main chamber 101a just prior to a next sterilant gas injection phase. Alternatively, the basket may remain outside the main chamber 101a after a last sterilant gas charge occurs. Thereby, a sterilant residual kill to the BI test samples may be reduced during a single or multiple sterilant gas injection resistometer study.

The remote device 125b may include a memory 130b and a processor 132b for storing and executing, respectively, a module 131b. The module 131b, stored in the memory 130b as a set of computer-readable instructions, may be related to an application for implementing at least a portion of the system for evaluate inactivation kinetics of a biological indicator 100b. As described in detail herein, the processor 132b may execute the module 131b to, among other things, cause the processor 132b to receive, generate, and/or transmit data with the network 135b, the resistometer 105b, and/or the printer 121b. Additionally, the processor 132b may execute at least a portion of the module 131b to, for example, cause the processor 132b to determine a D-value using enumeration data that is determined from a slope of a linear regression fit (e.g., linear regression fit of FIG. 5B) of a logarithm of a BI population versus an exposure time. The processor 132b may execute at least a portion of the module 131b to, for example, cause the processor 132b to determine MPN data a D-value through industry standard calculations defined in standards such as (but not limited to) the Stumbo Murphy Cochran Method and the Holcomb Spearman Karber Method.

The remote device 125b may also include a user interface 126b which may be any type of electronic display device, such as touch screen display, a liquid crystal display (LCD), a light emitting diode (LED) display, a plasma display, a cathode ray tube (CRT) display, or any other type of known or suitable electronic display along with a user input device. An associated user interface may exhibit a user interface display (e.g., any user interface 111a, 126a of FIG. 1A, any of the graphs of FIGS. 2-5B, etc.) which may, for example, depict a user interface for implementation of at least a portion of the system for evaluate inactivation kinetics of a biological indicator 100b.

The remote device 125b may also include a biological indicator inactivation related database 127b and a network interface 133b. The biological indicator inactivity database 127b may, for example, store biological indicator related data, etc. The network interface 133b may be configured to facilitate communications, for example, between the remote device 125b and the network 135b via any wireless communication network 137b, including for example: TLS v1.2 Cellular, CSV/JSON Output, TLS v1.2 REST API, a wireless LAN, MAN or WAN, WiFi, TLS v1.2 WiFi, the Internet, or any combination thereof. Moreover, a remote device 125b may be communicatively connected to any other device via any suitable communication system, such as via any publicly available or privately owned communication network, including those that use wireless communication structures, such as wireless communication networks, including for example, wireless LANs and WANs, satellite and cellular telephone communication systems, etc.

Turning to FIG. 1C, a system to evaluate inactivation kinetics of a biological indicator 100c may include a resistometer 105c having a user interface generation module 123c, a resistometer control data receiving module 124c, a resistometer sensor data receiving module 125c, a resistometer control module 126c, a biological indicator data generation module 127c, and a biological indicator data transmission module 128c, for example, stored on a memory 122c as a set of computer-readable instructions. In any event, the modules 123c-128c may be similar to, for example, the module 123b of FIG. 1B.

With reference to FIG. 1D, a method of implementing a resistometer 100d may be implemented by a processor (e.g., processor 124b of FIG. 1B) executing, for example, at least a portion of the module 124b of FIG. 1B or, at least a portion of the modules 123c-128c of FIG. 1C. In particular, processor 124b may execute the user interface generation module 123c to cause the processor 124b to, for example, generate a user interface display (block 123d). Any given user interface display may, for example, enable an individual to: control the resistometer 105c, evaluate inactivation kinetics of a biological indicator, view displays (e.g., any user interface 111a, 126a of FIG. 1A, any of the graphs of FIGS. 2-5B, etc.) related to evaluating inactivation kinetics of a biological indicator, etc.

Processor 124b may execute the resistometer control data receiving module 124c to cause the processor 249a to, for example, receive resistometer control data (block 124d). Processor 124b may execute the resistometer sensor data receiving module 125c to cause the processor 249a to, for example, receive resistometer sensor data (block 125d). Processor 124b may execute the resistometer control module 126c to cause the processor 249a to, for example, control the resistometer based on the resistometer sensor data and the resistometer control data (block 126d). Processor 124b may execute the biological indicator data generation module 127c to cause the processor 249a to, for example, generate biological indicator data based upon the resistometer sensor data, the resistometer control data, resistometer clock data, etc. (block 127d). Processor 124b may execute the biological indicator data transmission module 128c to cause the processor 249a to, for example, transmit biological indicator data to a remote device 125a, 125b and/or a printer 121a, 121b (block 128d).

Turning to FIG. 1E, a system to evaluate inactivation kinetics of a biological indicator 100e may include a remote device 125e having a user interface generation module 131e, a biological indicator data receiving module 132e, a resistometer sensor data receiving module 133e, an enumeration-D value data generation module 134e, a fraction-negative-D value data generation module 135e, a biological indicator inactivation profile data generation module 136e, a biological indicator inactivation profile extrapolation data generation module 137e, an enumeration-linear survivor curve data generation module 138e, a fraction-negative-linear survivor curve data generation module 139e, a linear biological indicator survivor curve data generation module 140e, and a biological indicator D value deviation data generation module 141e, for example, stored on a memory 130e as a set of computer-readable instructions. In any event, the modules 131e-141e may be similar to, for example, the module 131e of FIG. 1E.

With reference to FIG. 1F, a method of implementing a remote device 100f may be implemented by a processor (e.g., processor 132b of FIG. 1B) executing, for example, at least a portion of the module 131b of FIG. 1B or, at least a portion of the modules 131e-141e of FIG. 1E. In particular, processor 132b may execute the user interface generation module 131e to cause the processor 132b to, for example, generate a user interface display (block 131f). Any given user interface display may, for example, enable an individual to: control the resistometer 105c, evaluate inactivation kinetics of a biological indicator, view displays (e.g., any user interface 111a, 126a of FIG. 1A, any of the graphs of FIGS. 2-5B, etc.) related to evaluating inactivation kinetics of a biological indicator, etc.

Processor 132b may execute the biological indicator data receiving module 132e to cause the processor 132b to, for example, receive biological indicator data (block 132f). Processor 132b may execute the resistometer sensor data receiving module 133e to cause the processor 132b to, for example, receive resistometer sensor data (block 133f). Processor 132b may execute the enumeration-D value data generation module 134e to cause the processor 132b to, for example, generate enumeration-D value data via an enumeration method (block 134f). Processor 132b may execute the fraction-negative-D value data generation module 135e to cause the processor 132b to, for example, generate fraction-negative-D value data via a fraction-negative method (block 135f). Processor 132b may execute the biological indicator inactivation profile data generation module 136e to cause the processor 132b to, for example, generate biological indicator inactivation profile data (block 136f). Processor 132b may execute the biological indicator inactivation profile extrapolation data generation module 137e to cause the processor 132b to, for example, generate biological indicator inactivation profile extrapolation data (block 137f). Processor 132b may execute the enumeration-linear survivor curve data generation module 138e to cause the processor 132b to, for example, generate enumeration-linear survivor curve data (block 138f). Processor 132b may execute the fraction-negative-linear survivor curve data generation module 139e to cause the processor 132b to, for example, generate fraction-negative-linear survivor curve data (block 139f). Processor 132b may execute the linear biological indicator survivor curve data generation module 140e to cause the processor 132b to, for example, generate linear biological indicator survivor curve data (block 140f). Processor 132b may execute the biological indicator D value deviation data generation module 141e to cause the processor 132b to, for example, generate biological indicator D value deviation data (block 141f).

In lieu of an apparatus that takes BI viable spore counts data directly from chamber readouts, processor 132b may receive viable spore counts data from enumeration and Fraction negative tests, and may provide individual or combined survival curve, D Value, linearity information for the BI. In this case, BI viable spore counts data may be entered manually by a user from enumeration data (block 132f). Alternatively, the processor 132b may receive BI sterility test results entered manually by, for example a user, to determine viable spore counts from Fraction Negative data (i.e., a user may enter exposure time and additional processing conditions information for each BI test group (block 133f) and block 135f may not be included). BI viable spore counts data may be entered manually by user from enumeration data (block 132f). Alternatively, or additionally, BI sterility test results may be entered manually by user to determine viable spore counts from Fraction Negative data (block 133f). A user may enter exposure time and additional processing conditions information for each BI test group (block 135f).

With reference to FIG. 1G, a method of implementing a remote device 100f may be implemented by a processor (e.g., processor 132b of FIG. 1B) executing, for example, executing, for example, at least a portion of the module 131b of FIG. 1B or, at least a portion of the modules 131e-141e of FIG. 1E. In particular, processor 106b may execute at least a portion of the module 131b to cause the processor 132b to, for example, generate a user interface display (block 108g). Any given user interface display may, for example, enable an individual to evaluate inactivation kinetics of a biological indicator.

Processor 132b may execute at least a portion of the module 131b to cause the processor 132b to, for example, receive biological indicator viable spore counts data (block 109g). The biological indicator viable spore counts data may be representative of, for example, enumeration testing results associated with biological indicators. Processor 132b may execute at least a portion of the module 131b to cause the processor 132b to, for example, receive biological indicator data (block 110g). The biological indicator data may be representative of, for example, sterility test results 102a associated with biological indicators.

Processor 132b may execute at least a portion of the module 131b to cause the processor 132b to, for example, generate viable spore counts and/or survival curve data (block 111g). The viable spore counts and/or survival curves may be based on, for example, enumeration study data and/or data resulting from performing a fraction negative method. Processor 132b may execute at least a portion of the module 131b to cause the processor 132b to, for example, generate D values data (block 112g). The D values data may be representative of, for example, at least one survival curve for a biological indicator.

Processor 132b may execute at least a portion of the module 131b to cause the processor 132b to, for example, combine multiple biological indicator data sets (block 113g). For example, the processor 132b may combine multiple biological indicator data sets, and may construct survival curves based on the combined biological indicator data sets. Processor 132b may execute at least a portion of the module 131b to cause the processor 132b to, for example, generate biological indicator inactivation profile extrapolation data (block 114g). For example, the processor 132b may generate biological indicator inactivation profile extrapolation data based on the combined biological indicator data sets. Processor 132b may execute at least a portion of the module 131b to cause the processor 132b to, for example, calculate D Values and assess linearity of an individual or combined survival curves (block 115g).

With reference to FIG. 2, a graph 200 of an inactivation profile for a biological indicator based on D values determined using a direct enumeration method and a fraction-negative method and a single sterilant gas injection phase may include a first region 201 and a second region 202. An example Biological Indicator inactivation profile is illustrated to demonstrate challenges for characterizing linearity over a full range of inactivation for certain gaseous “surface” sterilization modalities.

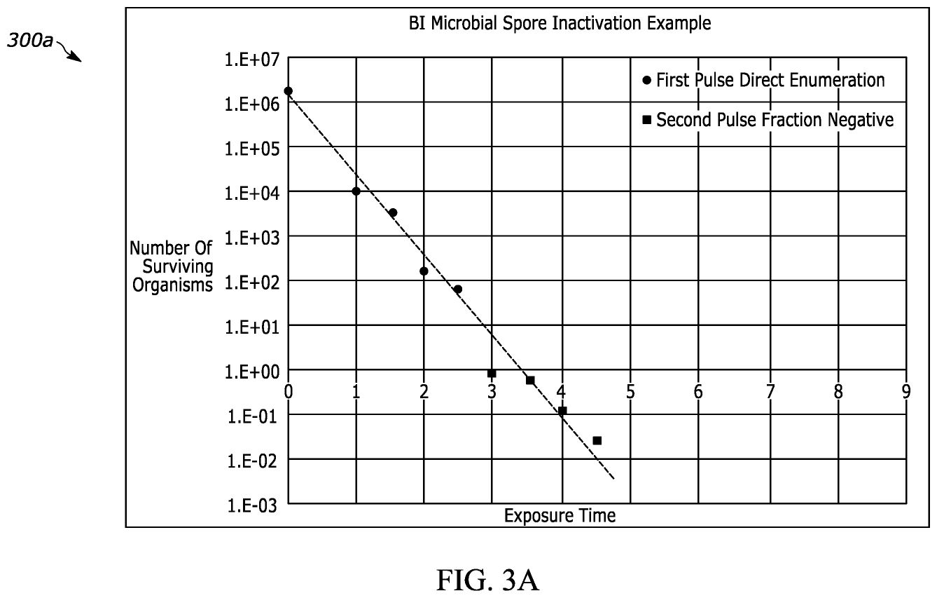

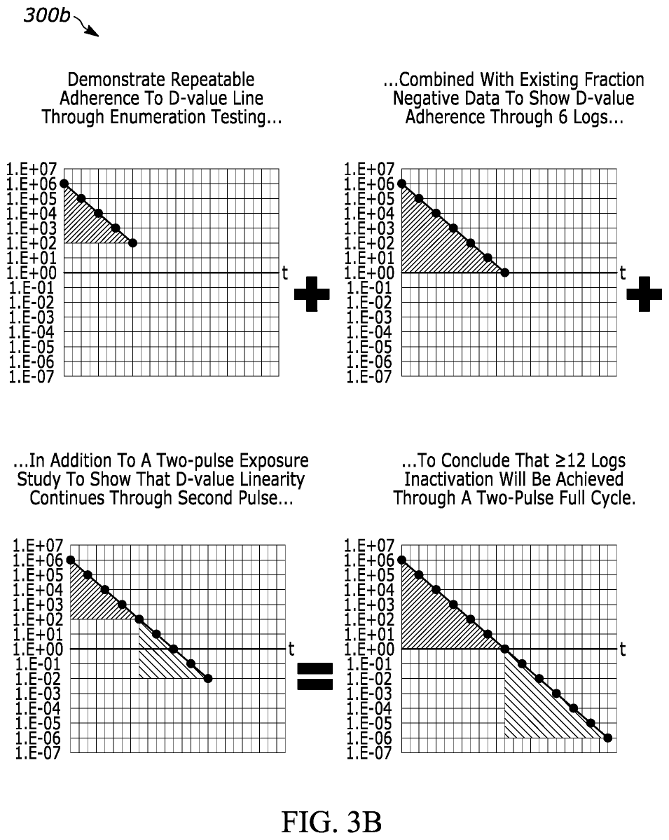

Turning to FIGS. 3A and 3B, graphs 300a, 300b illustrate an inactivation profile for a biological indicator based determined using a direct enumeration method and a fraction-negative method and multiple sterilant gas injection phases. Graph 300a reflects a theoretical BI inactivation profile using combined data from, for example, enumeration results obtained from BIs processed in, for example, a one gas charge resistometer cycle and data from, for example, fraction negative MPN results obtained from BIs processed in, for example, a two charge resistometer cycle. Graph 300b represents a method to demonstrate linearity and extrapolation to 12 spore log reduction using combined enumeration and fraction negative MPN results from BIs processed in one- and two-charge resistometer cycles using theoretical data.

A method 100d and/or 100f may, for example, characterize resistance and the inactivation profile of BIs used for gaseous sterilization modalities that utilize multiple sterilant charges per cycle. For example, resistometer study conditions with multiple gas charges may be employed to collect data points to establish a survival curve using both enumeration and fraction negative most probable number (MPN) calculated spore counts. A method 100d and/or 100f may, for example, characterizes the resistance of the BIs via the following approach: 1) a data collection part 1: 1a) BI samples may be processed in resistometer conditions where only one gas charge is employed and where conditions may be favorable to achieve a spore log reduction from, for example, 10⁶ to 10²; 1b) Enumeration testing is performed to collect viable spore count data points on the survival curve between populations of, for example, 10⁶ and 10² for BI samples; 1c) fraction negative and Most Probable Number assessment on, for example, 50 BIs per test group (between 20-100 BIs, most preferably 50 BIs) to achieve data points on the survival curve between populations of 10² to 10^{0; 2}) a data collection part 2: 2a) Extrapolating the inactivation profile using the D Value from the survival curve data points collected above and assuming linear inactivation kinetics, determine the sterilant gas dwell time needed to achieve a spore population of, for example, 10² and 10⁻². Design each subsequent resistometer cycle runs to have 2 sterilant gas charges-one charge providing the sterilant exposure conditions required to achieve, for example, a 4 spore log reduction from, for example, 10⁶ to 10²; and another equivalent charge to provide additional sterilant exposure conditions needed to achieve a spore log reduction from, for example, 10² to 10⁻². The second gas charge may be designed to maintain the starting chamber sterilant concentration levels achieved during the first sterilant gas charge; 2b) Process BI samples with, for example, 50 BIs per test group (between 20-100 BIs, most preferably 50 BIs) in resistometer processing conditions of only one gas charge targeting a spore log reduction of, for example, 4 (targeting a population of, for example 10²); 2c) Process BI samples with at least, for example, 50 samples per test group in subsequent and separate runs utilizing resistometer processing conditions of 2 gas charges and pull BI test groups at targeted extrapolated spore log reduction points of 5 (targeting a population of 10¹), 6 (targeting 10⁰), 7 (targeting 10⁻¹) and 8 (targeting 10⁻²), Or other similarly calculated intervals to achieve 2-8, or most preferably 3-5, data points in the, for example, 10² to 10⁻² spore population range; 2d) Perform BI sterility testing, fraction negative and Most Probable Number calculations to collect viable spore count data points on the survival curve for a targeted population of 10² for BIs exposed to resistometer conditions of only one gas charge; and between populations of, for example, 10¹ to 10⁻² for BIs exposed to resistometer conditions of two gas charges; 3) Combine the following spore count data to construct the survival curve over the entire range of inactivation; 3a) enumeration spore count data for data points of spore counts from, for example, 10⁶ to 10²; 3b) fraction negative data Most Probable Number spore count data points of spore counts from, for example, 10² to 10⁻²; 3c) Assess the inactivation kinetics of the BIs by evaluating the characteristics of the survivor curve and its linearity; and demonstrate continued linearity and maintenance of D Value through multiple/additional gas charges

A survival curve may, thereby, be characterized as having the logarithm of the surviving population decreasing linearly with the exposure time, through the Direct Enumeration region and into the Fraction Negative region. Multiple NO₂ gas charges per resistometer cycle may improve continued linearity past 6 logs for a BI resistance and inactivation profile. The overkill approach for validation of gaseous sterilization processes as described in ISO 14937, in which one extrapolates from 6 to 12 log reduction in a full cycle after achieving all kill for internal process challenge devices (iPCDs) in a half cycle may subsequently be performed. Linearity may be achieved via both new enumeration and existing fraction negative data for the first 4 log reduction from 10⁶ to 10² under 1 gas charge conditions. This data may be combined with existing certificate of analysis (CoA) data to demonstrate a 6 spore log reduction via one gas charge.

The above description describes various devices, assemblies, components, subsystems and methods for evaluating inactivation kinetics of a biological indicator (e.g., combination product, biosimilars, biologics, etc.) and medical devices. The devices, assemblies, components, subsystems, methods can further comprise or be used with, for example, a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir within the pre-filled syringe for example. In some instances, the reservoir is a primary container that is either filled or pre-filled for treatment with the drug. The primary container can be a vial, a cartridge or a pre-filled syringe. In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific lgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIIa receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGB mAb (MDX-1307); anti-mesothelin dsFv-PE38 conjugate (CAT-5001); anti-PD1mAb (MDX-1106 (ONO-4538)); anti-PDGFRa antibody (IMC-3G3); anti-TGFß mAb (GC-1008); anti-TRAIL Receptor-2 human mAb (HGS-ETR2); anti-TWEAK mAb; anti-VEGFR/FIt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (lgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein (a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BITE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BITE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A computer-implemented method for evaluating inactivation kinetics of a biological indicator using data obtained from studies utilizing a resistometer, wherein the resistometer is configured to implement a gaseous sterilization process, the method comprising:
receiving, using data obtained from studies utilizing the resistometer, a first set of biological indicator data from test samples exposed to NO₂ sterilization processing conditions of at least one first sterilant gas injection phase, wherein the first set of biological indicator data is representative of viable spore counts of the biological indicator;
determining D values, using an enumeration method for determining viable spore counts, based on the first set of biological indicator data;
receiving, using data obtained from studies utilizing the resistometer, a second set of biological indicator data during at least one additional sterilant gas injection phase, wherein the second set of biological indicator data is representative of viable spore counts of the biological indicator;
determining D values, using a fraction negative method for determining viable spore counts, based on the second set of biological indicator data; and
establishing an inactivation profile of the biological indicator based on combined viable spore counts determined from an enumeration fraction-negative-method.

2. The method of claim 1, wherein the inactivation profile of the biological indicator is representative of a half sterilization cycle, and the method further comprising:
extrapolating the inactivation profile of the biological indicator to be representative of a full sterilization cycle, wherein the full sterilization cycle includes double the exposure conditions as compared to the half sterilization cycle.

3. The method of claim 2, wherein the full sterilization cycle results in at least a 12-spore log reduction in viable spore counts for the biological indicator.

4. The method of claim 1, wherein the at least one first sterilant gas injection phase is associated with a first 4 log reduction in viable spore counts of the biological indicator.

5. The method of claim 1, wherein the second sterilant gas injection phase maintains starting resistometer chamber sterilant concentration levels achieved during the first sterilant gas injection phase.

6. The method of claim 1, wherein the biological indicators are subject to an additional sterilant gas injection phase, and had experienced 4 log reduction in viable spore counts compared to its starting population prior to this additional gas injection phase.

7. The method of claim 1, wherein the gaseous sterilizing process exhibits non-linear inactivation kinetics for the biological indicator.

8-14. (canceled)

15. A computer-readable medium storing computer-readable instructions that, when executed by a processor, cause the processor to evaluate inactivation kinetics of a biological indicator using data from studies that utilized a resistometer, wherein the resistometer is configured to implement a gaseous sterilization process, the computer-readable medium comprising:
a first set of biological indicator data receiving module that, when executed by a processor, causes the processor to receive a first set of biological indicator data obtained from test samples processed under at least one first sterilant gas injection phase, wherein the first set of biological indicator data is representative of viable spore counts of the biological indicator;
an enumeration-D values data generation module that, when executed by a processor, causes the processor to generate D values from enumeration viable spores counts data, implementing an enumeration method for determining viable spore counts, based on the first biological indicator data;
a second set of biological indicator data receiving module that, when executed by a processor, causes the processor to receive a second set of biological indicator data during at least one second sterilant gas injection phase, wherein the second biological indicator data is representative of viable spore counts of the biological indicator;
a fraction-negative-D values data generation module that, when executed by a processor, causes the processor to generate fraction-negative-D values, implementing a fraction negative method for determining viable spore counts, based on the second set of biological indicator data; and
an inactivation profile data generation module that, when executed by a processor, causes the processor to establish an inactivation profile of the biological indicator based on the enumeration-D values data and the fraction-negative-D values data.

16. The computer-readable medium of claim 15, wherein the inactivation profile of the biological indicator is representative of a half sterilization cycle, and the computer-readable medium further comprising:
an inactivation profile extrapolation data generation module that, when executed by a processor, causes the processor to extrapolate the inactivation profile of the biological indicator to be representative of a full sterilization cycle, wherein the full sterilization cycle includes exposure conditions double the exposure conditions of the half sterilization cycle.

17. The computer-readable medium of claim 16, wherein the full sterilization cycle results in a 12-spore log reduction in viable spore counts for the biological indicator.

18. The computer-readable medium of claim 15, wherein the at least one first sterilant gas injection phase is associated with a first 4 log reduction in viable spore counts of the biological indicator.

19. The computer-readable medium of claim 15, wherein the second sterilant gas injection phase maintains starting resistometer chamber sterilant concentration levels achieved during the first sterilant gas injection phase.

20. The computer-readable medium of claim 15, wherein the second sterilant gas injection phase starts at the 4 log reduction in viable spore counts of the biological indicator.

21. The computer-readable medium of claim 15, wherein the gaseous sterilizing process exhibits linear inactivation kinetics for the biological indicator.

22-28. (canceled)

29. A system to evaluate inactivation kinetics of a biological indicator using a resistometer that is configured to implement a gaseous sterilization process, the system comprising:
a time clock;
a resistometer chamber pressure sensor;
a resistometer chamber temperature sensor;
at least one resistometer chamber sterilant concentration level sensor;
a first set of biological indicator data receiving module stored on a memory that, when executed by a processor, causes the processor to receive a first set of biological indicator data during at least one first sterilant gas injection phase, wherein the first biological indicator data is representative of viable spore counts of the biological indicator;
an enumeration-D values data generation module stored on a memory that, when executed by a processor, causes the processor to generate enumeration-D values data, implementing an enumeration method for determining viable spore counts, based on the first biological indicator data;
a second set of biological indicator data receiving module stored on a memory that, when executed by a processor, causes the processor to receive a second set of biological indicator data during at least one second sterilant gas injection phase, wherein the second biological indicator data is representative of viable spore counts of the biological indicator;
a fraction-negative-D values data generation module stored on a memory that, when executed by a processor, causes the processor to generate fraction-negative-D values, implementing an fraction negative method for determining viable spore counts, based on the second set of biological indicator data; and
an inactivation profile data generation module stored on a memory that, when executed by a processor, causes the processor to establish an inactivation profile of the biological indicator based on the enumeration-D values and the fraction-negative-D values.

30. The system of claim 29, wherein the inactivation profile of the biological indicator is representative of a half sterilization cycle, and the system further comprising:
an inactivation profile extrapolation data generation module stored on a memory that, when executed by a processor, causes the processor to extrapolate the inactivation profile of the biological indicator to be representative of a full sterilization cycle, wherein the full sterilization cycle includes exposure conditions double the exposure conditions of the half sterilization cycle.

31. The system of claim 30, wherein the full sterilization cycle results in a 12-spore log reduction in viable spore counts for the biological indicator.

32. The system of claim 29, wherein the at least one first sterilant gas injection phase is associated with a first 4 log reduction in viable spore counts of the biological indicator.

33. The system of claim 29, wherein the second sterilant gas injection phase (a) maintains starting resistometer chamber sterilant concentration levels achieved during the first sterilant gas injection phase and/or (b) starts at the 4 log reduction in viable spore counts of the biological indicator.

34. (canceled)

35. (canceled)

36. The system of claim 29, where the at least one resistormeter chamber sterilant concentration level sensor includes at least one of: a nitrogen dioxide (NO₂) sensor, a hydrogen peroxide (H₂O₂) sensor, an ethylene oxide (C₂H₄O) sensor, or a moist heat sensor.

37-77. (canceled)
